(19) United States
(12) Patent Application Publication
(10) Pub. No.: US 2023/0091616 A1
(43) Pub. Date: Mar. 23, 2023

(54) Method and Apparatus for Disposal of Sanitary Waste

(71) Applicant: Farnaz Zand, Burbank, CA (US)

(72) Inventor: Farnaz Zand, Burbank, CA (US)

(21) Appl. No.: 17/746,538

(22) Filed: May 17, 2022

Related U.S. Application Data
(60) Provisional application No. 63/247,618, filed on Sep. 23, 2021.

(51) Int. Cl. A61F 13/551

(52) U.S. Cl. CPC A61F 13/5512; A61F 13/5515; A61F 2013/55155; A61F 2013/55125

(57) ABSTRACT

A method and apparatus for disposing of sanitary waste such as used adult and children diapers, sanitary pads, napkins, incontinence sheets, and like waste products.

### CROSS-REFERENCE TO RELATED APPLICATIONS

This is a Non-Provisional application claiming the benefit of co-pending Provisional Application No. 63/247,618 filed Sep. 23, 2021.

### STATEMENT REGARDING FEDERALLY SPONSORED RESEARCH OR

DEVELOPMENT: Not Applicable

### INCORPORATION-BY-REFERENCE OF MATERIAL SUBMITTED ON A

COMPACT DISC: Not Applicable

### BACKGROUND OF THE INVENTION

### Field of the Invention

The present invention relates generally to a method and apparatus for disposing of sanitary waste such as used adult and children diapers, sanitary pads, napkins, incontinence sheets, and like waste products.

### Discussion of the Prior Art

Sanitary waste disposal has become an increasing problem. When soiled sanitary items are disposed into the trash, they can threaten the health of sanitation workers and others who may come in contact with the waste matter. Clearly, fecal matter and human waste has the potential to be dangerous and disposing the soiled diaper and other sanitary items is a very serious matter. Additionally, if landfills are not properly constructed, bacteria from fecal matter can leech into the groundwater and contaminate it. In fact, over 100 viruses can be detected in human excrement—many of which can live for months outside the body. Some of the bacteria and viruses which may be present in human fecal matter comprise Gastrointestinal viruses, Hepatitis, E. coli, Salmonella, Norovirus, Polio and Listeria. Additionally, soiled diapers containing fecal matter can be the source of pathogens that cause gastrointestinal illness. Children in child care centers commonly excrete intestinal pathogens even if not presenting symptoms. For example, noroviruses can be shed in the feces of children for at least 25 days after symptoms have stopped. Similarly, rotavirus can be shed for 25-57 days after the onset of diarrhea in a child. Continued shedding of pathogens in the feces of asymptomatic children can increase the transmission to healthy individuals. Surfaces play an important role in the spread of pathogens. Noroviruses can survive up to 42 days at room temperature when dried onto a surface.

In recent years, the government published rules requiring companies to provide disposal wrappers for diapers and sanitary pads and to pay for waste management systems directed to the exploration of recyclable alternatives. These rules also make it obligatory for the manufacturers, brand owners or marketing companies of sanitary napkins and diapers to provide a pouch or wrapper for safe disposal of used sanitary napkin or diapers along with the package sanitary products. However, history has shown that many companies do not provide wrappers for certain products and that when provided, are often badly designed and frequently fail.

### SUMMARY OF THE INVENTION

The present invention relates generally to the disposal of sanitary waste. More particularly, the invention concerns an apparatus for disposing of sanitary waste such as used adult and infant diapers, sanitary pads, napkins, incontinence sheets and like waste products and of a method of using the apparatus. In one form of the invention, the apparatus comprises a fluid absorbent body, a decorative covering encapsulating at least a portion of the body and a pouch disposed between the fluid absorbent body and the covering. The pouch has an interior chamber and a slit providing access to the chamber. Disposed within the interior chamber of the pouch is an expandable, sealable plastic bag. To assist the user in removing the plastic bag from the pouch, a pull strip is connected to the bag.

With the forgoing in mind, it is an object of the present invention to provide a disposable waste product such as a diaper and sanitary pad with attached sealable waste bag.

Another object of the present invention is to provide such a disposable waste product with a pocket or pouch containing an expandable plastic waste bag that is securely connected to a selected surface of the product.

Still another object of the invention to provide a disposable waste product of the character described in the preceding paragraph in which the expandable plastic waste bag is large enough to fully encapsulate the soiled components of the disposable waste product.

Yet another object of the present invention is to provide a disposable waste product of the aforementioned character in which the expandable plastic waste bag has a sealable upper end.

### BRIEF DESCRIPTION OF THE DRAWINGS

FIG. 1 is a generally perspective view partly broken away to show internal construction of one form of the disposable waste product of the invention.

FIG. 2 is a generally perspective view similar to FIG. 1, but illustrating an initial step in the method of the invention for disposing of the soiled fluid absorbent body of the waste product.

FIG. 3 is a generally perspective view similar to FIG. 2, but illustrating the next step of the method of the invention for disposing the soiled fluid absorbent body of the waste product.

FIG. 4 is a generally perspective view similar to FIG. 3, illustrating a further step in the method of the invention for disposing the soiled fluid absorbent body of the waste product.

FIG. 5 is a generally perspective view similar to FIG. 4, illustrating the final step in the method of the invention for disposing the soiled fluid absorbent body of the waste product.

FIG. 6 is a generally perspective view of another form of the disposable waste product of the invention.

### DESCRIPTION OF THE INVENTION

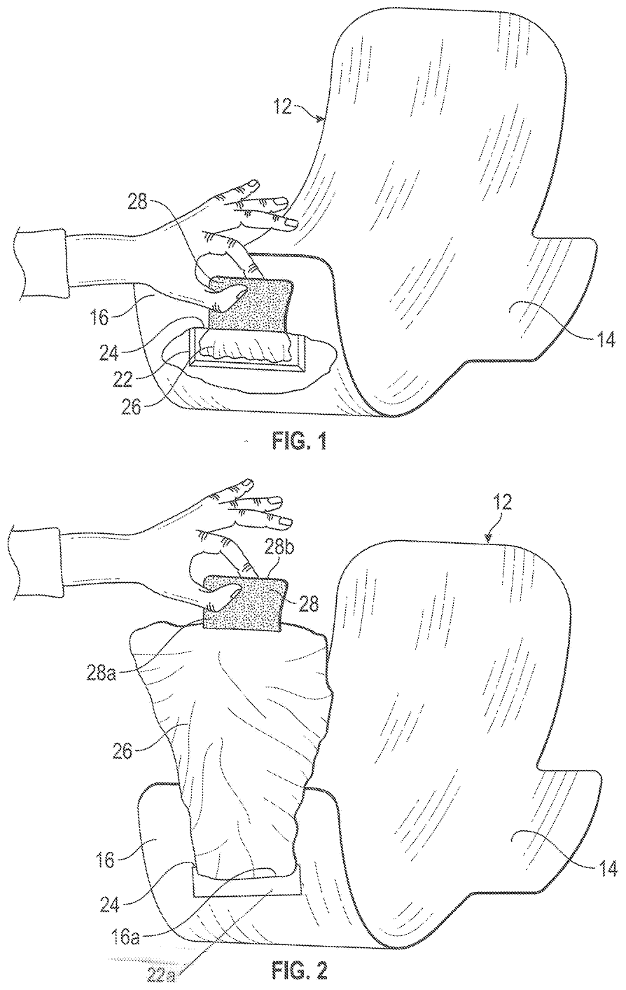

Referring to the drawings and particularly to FIG. 1, one form of the disposable waste product of the invention for disposing of sanitary waste such as used adult and infant diapers, sanitary pads, napkins, incontinence sheets and like waste products is there shown. In FIG. 1, the apparatus is shown as a diaper structure 12 having a fluid absorbent body 14 covered by a decorative covering 16. Disposed between covering 16 and fluid absorbent body 14 is a generally rectangular shaped pouch 22. Pouch 22 is provided with a slit 24 for allowing entrance to an egress from the pouch. For a purpose presently to be described, an expandable plastic bag 26 is removably disposed within the pouch 22. To permit the controlled removal of the plastic bag from the pouch 22 in the manner shown in FIG. 2 of the drawings, a pull strip 28 is provided. Pull strip, which is receivable within slit 24 and within a slit 16A provided in covering 16, has a first end 28a connected to the plastic bag and a second end 28b (FIG. 2) extending outwardly of slit 24.

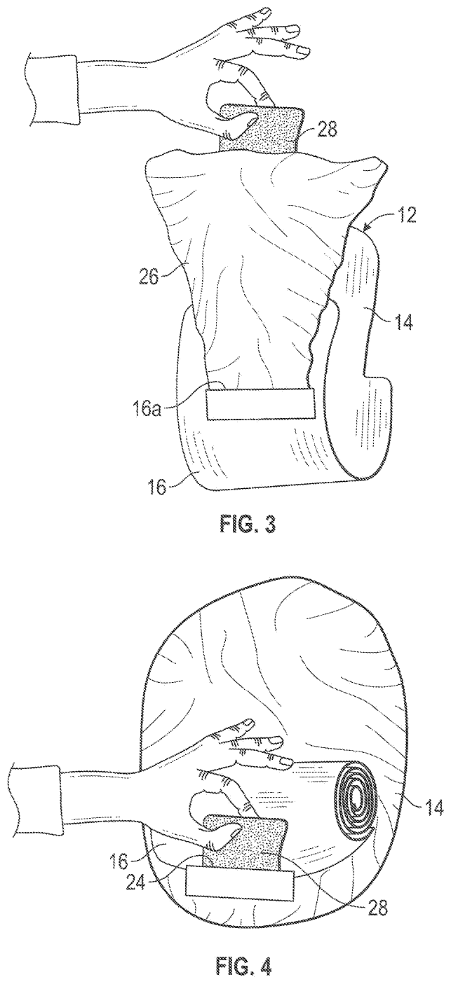

In using the disposable waste product of the invention, the first step in the preferred method of the invention comprises wrapping the inner side of the disposable waste product about the user's bottom and between its legs. This done, upon discovering that the fluid absorbent body 14 of the disposable waste product has become soiled, the next step in the method of the invention is to remove the disposable waste product from the diapered infant or adult. This done, the pull strip 28 is grasped and pulled outwardly to remove the expandable plastic bag 26 from the pouch 22 (see FIG. 3). Next, in the manner illustrated in FIG. 4, the soiled fluid absorbent body of the disposable waste product is folded back upon itself and inserted into the open end of the plastic bag 26.

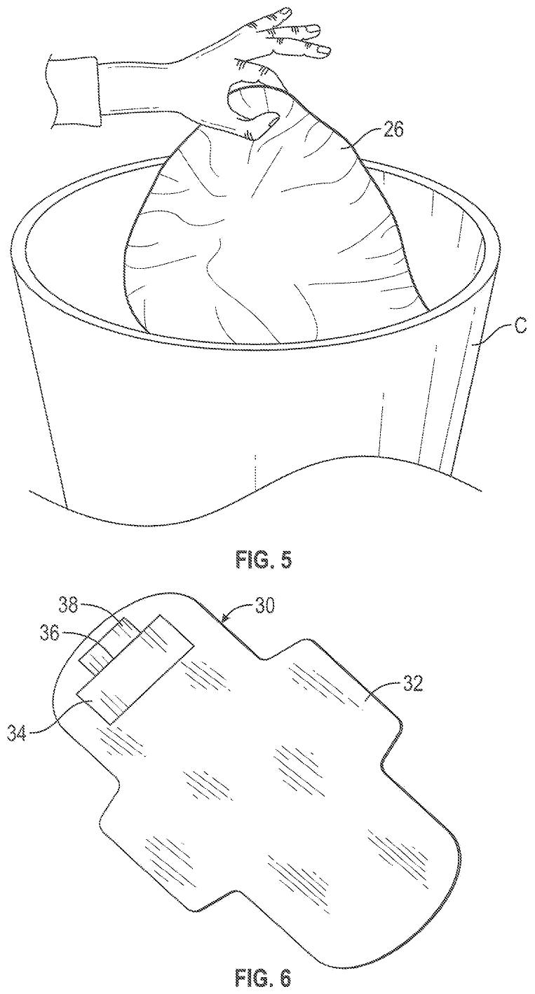

With the soiled fluid absorbent body of the disposable waste product fully encapsulated within the plastic bag in the manner shown in FIG. 5, the next step in the method of the invention is to seal the open end of the plastic bag by tying the open end of the bag in the manner illustrated in FIG. 5 of the drawings. The final step in the method of the invention is to dispose of the sealed plastic bag into an appropriate disposal container “C”.

Turning now to FIG. 6 of the drawings, another form of the disposable waste product of the invention is there shown and generally designated by the numeral 30. This form of the invention, which shows the disposable waste product of the invention for disposing of sanitary pads, is similar in many respects to the disposable waste product shown in FIGS. 1-5. Product 30 has a fluid absorbent body 32. Affixed to body 32 is a generally rectangular shaped pouch 34. As was the case with pouch 22, it is provided with a slit 36 for allowing entrance to an egress from the pouch. As before, an expandable plastic bag (not shown) is removably disposed within the pouch 34. To permit the controlled removal of the plastic bag from the pouch 34, a pull strip 38 is provided. As in the previously described embodiment, pull strip, which is receivable within slit 36, is connected to the plastic bag.

In using this latest form of the invention, upon discovering that the fluid absorbent body 32 of the disposable waste product has become soiled, the initial step in this latest method of the invention now to be described is to remove the expandable plastic bag from the pouch 34. This is done by grasping the pull strip 38 and pulling outwardly with sufficient force to remove the bag from the pouch. Next the soiled fluid absorbent body of the disposable waste product is folded back upon itself and inserted into the open end of the plastic bag.

With the soiled fluid absorbent body of the disposable waste product fully encapsulated within the plastic bag in the manner shown in FIG. 5, the next step in the method of the invention is to seal the open end of the plastic bag by tying the open end of the bag in the manner illustrated in FIG. 5 of the drawings. The final step in the method of the invention is to dispose of the sealed plastic bag into an appropriate disposal container “C”.

Having now described the invention in detail in accordance with the requirements of the patent statutes, those skilled in this art will have no difficulty in making changes and modifications in the individual parts or their relative assembly in order to meet specific requirements or conditions. Such changes and modifications may be made without departing from the scope and spirit of the invention as set forth in the following claims.

I claim:

1. A disposable waste product having a yieldably deformable, fluid absorbent body, a covering at least covering a portion of said fluid absorbent body, a pouch disposed between said fluid absorbent body and said covering, said pouch having an interior chamber and a slit providing access to said interior chamber of said pouch; and an expandable, sealable plastic bag disposed within said interior chamber of said pouch.

2. The disposable waste product as defined in claim 1 further including a pull strip connected to said plastic bag.

3. The disposable waste product as defined in claim 1 in which said sealable plastic bag has an expandable interior chamber.

4. The disposable waste product as defined in claim 1 in which said covering is decorative.

5. A disposable waste product comprising:
(a) a yieldably deformable, fluid absorbent body;
(b) a decorative covering encapsulating at least a portion of said fluid absorbent body;
(c) a pouch disposed between said fluid absorbent body and said covering, said pouch having an interior chamber and a slit providing access to said interior chamber of said pouch;
(d) an expandable, sealable plastic bag disposed within said interior chamber of said pouch, said sealable plastic bag having an expandable interior chamber; and
(e) a pull strip connected to said plastic bag.

6. A method of using a disposable waste product of the character having an inner side and an outer side, a fluid absorbent body disposed between said inner side and said outer side, a pouch disposed between said outer side and said fluid absorbent body and an expandable, sealable plastic bag removably disposed within said pouch, said method including the steps of:
(a) wrapping the inner side of the disposable waste product about the user's bottom and between its legs;
(b) upon the fluid absorbent body of the disposable waste product being soiled, removing disposable waste product from the user;
(c) grasping the pull strip and pulling it outwardly to remove the expandable plastic bag from the pouch; and
(d) inserting the soiled fluid absorbent body of the disposable waste product into the plastic bag.

7. The method of using a disposable waste product as defined in claim 6, including the further step of sealing the plastic bag to produce a sealed plastic bag.
